(19) United States
(12) Patent Application Publication
(10) Pub. No.: US 2015/0060699 A1
(43) Pub. Date: Mar. 5, 2015

(54) AUTHENTICATION SYSTEMS EMPLOYING FLUORESCENT DIAMOND PARTICLES

(71) Applicant: Taaneh, Inc., New York, NY (US)

(72) Inventor: Andrew S. JANOFF, Princeton, NJ (US)

(21) Appl. No.: 14/477,704

(22) Filed: Sep. 4, 2014

Related U.S. Application Data
(60) Provisional application No. 61/873,686, filed on Sep. 4, 2013.
(60) Provisional application No. 61/926,854, filed on Jan. 13, 2014.

(51) Int. Cl. G01N 33/15; G01N 21/64; C09K 11/65

(52) U.S. Cl. CPC G01N 33/15; C09K 11/65; G01N 21/6447; G01J 3/28
USPC 250/459.1; 428/29; 423/446; 252/301.4 F; 250/216

(57) ABSTRACT

Authentication systems for products employing populations containing particles of diamonds that have fluorescent emissions of various wavelengths, intensities and durations are described. By varying the populations of diamond particles in products to be labeled, multiple different identification systems can be obtained permitting authentication taggants for large numbers of different products.

### CROSS-REFERENCE TO RELATED APPLICATIONS

This application claims priority to U.S. provisional patent application 61/873,686, filed on 4 Sep. 2013 and U.S. provisional patent application 61/926,854, filed on 13 Jan. 2014. The contents of said applications are incorporated herein in their entirety.

### TECHNICAL FIELD

The invention relates to the field of anti-counterfeiting systems that can be incorporated into various products. In particular, it relates to such systems that employ populations of diamond particles that when exposed to appropriate sources of electromagnetic radiation fluoresce at certain wavelengths with certain intensities for certain periods of time.

### BACKGROUND ART

The literature of systems for preventing counterfeiting in a wide variety of products is very extensive. Numerous approaches have been employed, including imprinting designs, adding colorants, electronic microchips, and a vast array of alternatives. A few of these are listed below, but this is far from a comprehensive survey of the entire field.

U.S. Pat. No. 7,394,997 describes a “consumable” having taggant nanoparticles which particles have a plurality of different characteristics of different categories. The focus of this application is on inks or toners designed to be compatible with specific printers.

A particularly important area for counterfeit detection is the pharmaceutical arena. Obviously, the harm caused by counterfeited drugs is significantly more serious than the use of an unauthorized toner or ink in a printer. Many strategies have been employed. A family of U.S. patents: U.S. Pat. No. 7,874,489; U.S. Pat. No. 8,220,716 and U.S. Pat. No. 8,458,475 describe compositions that are labeled by a product authentication code which is a signature array, where the signature array comprises information about the absolute counts or relative counts of entities of at least two distinct clusters of entities. The method thus relies on determining the numbers of individual elements in sets of populations.

U.S. publication 2001/0014131 suggests a method to identify pharmaceutical products by stamping patterns on their surfaces with lateral dimensions smaller than about 100 μ. A similar approach is described in US2010/0297228 as well as in US2010/0297027. US2009/0304601 describes a method for marking a composition for use in oral administration using color-inducing oxides in the composition. US2007/0259010 employs printed dosage forms with internal patterns that can be used for authentication, including letters, numbers and bar codes.

US2007/0048365 discloses edible coatings for pharmaceuticals that can be imprinted with codes that are machine-readable. US2006/0118739 describes pharmaceuticals that have luminescent markers with a spectral signature characteristic of the authentic product. U.S. Pat. No. 8,144,399 utilizes a complex optical image system for identification of genuine pharmaceutical products. U.S. Pat. No. 8,069,782 uses stamped patterns as identification for solid pharmaceuticals. U.S. Pat. No. 7,619,819 employs an optical system that utilizes diffraction gratings.

US2013/0072897 employs electromagnetic transmitters and receivers for determining identity of a drug reservoir. Visible radiation may also be used.

None of these systems employ diamond particles, which have the advantage of being completely inert and thus do not interfere with the desired properties of the product, such as the mode of action and pharmacokinetics and pharmacodynamics of pharmaceutical products. Diamond particles have no effect on absorption, distribution, metabolism or elimination (ADME) and are not toxic.

It has long been known that both natural and synthetic diamonds emit fluorescence. An early review by Walker, J., Rep. Prog. Phys. (1979) 42:1607-1654 describes in detail the excitation and emission characteristics of various types of diamonds having impurities such as boron and nitrogen. As noted by the reviewer, these are the most common impurities in diamond. Boron leads to utility in some instances in semiconductor applications. Nitrogen results in defects that permit excitation by both visible and infrared light as well as by UV light and corresponding emissions. This article explains an idealized symmetry of the Stokes shift whereby a lower energy light is symmetrically emitted from a higher frequency absorption. The transition where vibrational states are zero in both electronic ground and excited state can be discerned in the fluorescence spectrum as the zero phonon line (ZPL) which is characteristic of a particular Stokes shift and can be used to identify diamond.

In addition to the Stokes shift, single diamond nanoparticles also show two photon excitation patterns wherein two photons of infrared light result in emission of visible wavelengths. Chang, Y.-R., et al., Nature Nanotechnology (2008) 3:284-288, is one of a recent series of publications describing the production and imaging of fluorescent nanodiamonds. U.S. Pat. No. 8,168,413 also describes this method for preparing luminescent diamonds, which is done by irradiating diamond particles of 1 nm to 100 nm with high energy and heating the resultant. The diamonds claimed have oxidized surfaces and contain 5 ppm to 1,000 ppm color centers.

Alternative methods are described in US2010/0135890 which employs particles in the microparticle range. The production of nitrogen vacancy centers (NV centers) responsible for the fluorescence in these cases is also described by Baranov, P. G., et al., Small (2011) 7:1533-1537.

Numerous types of color centers have been described and exist in both natural and synthetic diamond particles.

TABLE 1

| | Excitation | Emission | ZPL |
|---|---|---|---|
| Publication/Source | λ max (nm) | λ max (nm) | (nm) |
| negative NV ^{a} | 560 | 700 | 637 |
| neutral NV ^{a} | 532 | | 575 |
| N—V—N (H3) ^{b} | | 531 | 503 |
| N₃ ^{c} | | (blue) | 415 |
| hydrogen enriched ^{d} | | (yellow) | |
| boron enriched ^{e} | | 636-666 | |
| two photon emission ^{a} | 1100 | 700 | |

^{a} U.S. Pat. No. 8,167,413
^{b} Yu, et al., JACS (2005) 127: 17604-17605
^{c} Chenko, et al., Nature (1977) 270: 414-144
^{d} Fritsch, et al., Genes & Geniol. (1992) 28: 35-42
^{e} Steed, J. W., J. Appl. Phys. (2003) 94: 3091-4009

In addition, U.S. publication 2012/0178099 describes counterpart carbon nanoparticles that fluoresce in the visible range. These are doped carbon particles (FCN's) that have fluorescent quantum yields in the range of 5-15% and emission colors at 455 nm (excitation at 350 nm), 480 nm (excitation at 400 nm), 520 nm (excitation at 400 nm), 540 nm (excitation at 450 nm) and 590 nm (excitation at 500 nm). Thus, five different combinations of excitation emission peaks are available.

These FCN particles are described as being capable of conjugation to biological molecules as are nanoparticle diamond complexes in US2010/0305309.

Importantly, in addition to these technical and precise parameters that can be associated with authentication systems of considerable sophistication, a simpler approach is permitted by virtue of the ability of commercially available diamonds to emit various colors upon excitation with ultraviolet (UV) light. A commonly available LED source which emits light at 360 nm (and is not harmful to the eyes) has been shown to elicit red, green, blue and IR fluorescence in commercially available diamond particles. Thus, a very straightforward authentication method can use combinations of these populations of diamonds in various ratios or simply alone, perhaps in a particular symbol or set of patterns.

A distinct advantage of the diamond particles of the invention is that they are not cytotoxic. Indeed, diamond particles are used in dental polishing, and various publications have indicated that they can be used without cytotoxicity in biological systems. Schrand, A. M., et al., J. Phys. Chem. (2007) 111:2-7 showed that nanodiamonds ranging in size from 2-10 nm were not cytotoxic to a variety of cell types. Mohan, N., et al., Nano. Lett. (2010) 10:3692-3699 showed that fluorescent nanodiamonds were stable and nontoxic in C. elegans.

In view of the benign and inert nature of diamond particles and in view of the variety of spectral characteristics that can be achieved and associated with specific populations of such particles, diamond particles provide an excellent system for authentication of various products including pharmaceutical products and other materials such as textiles, inks, paint, currency, cosmetics, luxury items, fragrances or food.

### DISCLOSURE OF THE INVENTION

The invention provides an authenticating system that is useful in a wide variety of products. The basis for this system is a population of particles of diamond that exhibit specific spectra or colors whereby suitable wavelengths, intensities and durations of emission are associated with a specific excitation wavelength of suitable intensity and duration, where previously determined emission spectral data are associated with the population. The excitation may be a one-photon or a two-photon excitation. In its simplest form, a prescribed form of an authentication system comprising these particles and corresponding emission spectral characteristics are associated with the product, the presence of which indicates the authenticity of the product per se. In some embodiments, the presence of the prescribed forms of the authentication system is verified by excitation by a specific wavelength of specific intensity and duration combined with the intensity and/or duration of emission at selected wavelengths. In one embodiment, by coding the wavelength, intensity and duration of the excitation energy and providing this to the user, the manufacturer will permit the user to verify the authenticity of the product on site or by submission to a service provider based on the resulting emission signature.

In the alternative, a single excitation wavelength may generate different emission wavelengths and intensities depending on the nature of the diamond particles in the composition. As noted above, an ultraviolet light source emitting 360 nm can elicit red, green, blue or infrared (IR) fluorescence depending on the collection of diamond particles employed. A random mixture of such diamonds can be separated into various colors of emission by flow cytometry. (“Colors” includes UV, visible and IR emissions.) In general, “color” refers to the nature of fluorescence emissions—e.g., “green” refers to green fluorescence. A homogeneous population of such particles will provide a single color, though the complete spectrum will be more complex.

Thus, in one aspect, the invention is directed to a method for providing authentication to product which method comprises combining said product with a prescribed form of an authentication system (composition) which contains at least one population of diamond particles wherein said particles exhibit fluorescence with a fluorescence maximum at a particular wavelength, and wherein the wavelength, intensity and duration of the fluorescence of said particles is dependent on the wavelength, duration and intensity of the excitation energy.

In order to provide a variety of possible identification patterns, it is also advantageous to use more than one homogeneous population of said particles so that a multiplicity of different authenticating labels can be generated by varying the proportion of these populations in the resulting product. The populations will differ in excitation and emission spectral data i.e. wavelength, intensity and duration. Variations in this pattern may be obtained by varying the wavelength, intensity and duration of the excitation energy. Thus, the invention is also directed to a method for providing authentication by combining the product with a prescribed authentication system containing at least two homogeneous populations of particles wherein the wavelength, intensity and duration of the excitation and emission fluorescence is unique to each different population.

Still another level of authentication can be provided by including as a portion of the taggant (or as all of the taggant) unseparated diamond mixtures, i.e., a heterogeneous population. These mixtures appear non-fluorescent to the naked eye due to the cancellation of the fluorescence of the various components and the complexity of their interaction. However, excitation in the visible (or UV) light will result in a characteristic infrared spectrum which is difficult to duplicate using any counterfeit labeling that is different from heterogeneous populations of diamond particles, since these may vary from one such population to another.

Thus, still another aspect of the invention is directed to substrates that are tagged entirely or in part with an unseparated mixture of fluorescent diamond nanoparticles. The invention also includes authenticating these substrates by determining an IR spectrum based on visible or UV excitation.

In still another embodiment, the invention includes substrates tagged with diamond particles which substrates are comprised primarily of hydrophilic solid components, but further include a hygroscopic hydrophobic component. Upon application of pressure, the hydration water associated with the hygroscopic component is expelled creating an environment wherein diamond particles are unevenly distributed among the hydrophilic components and the hydrophobic dehydrated hygroscopic component. This redistribution is characteristic of diamond particles and is difficult to duplicate with substitute fluorescent materials. Thus, materials of this composition are also included within the invention and their characteristic “speckled” appearance in the presence of the diamond particles they contain is helpful in ascertaining the authentic nature of the substrate.

In some important embodiments, the product is a pharmaceutical, especially a solid oral dosage form, but the invention is useful in a wide variety of products.

In one embodiment, a population of particles that has a distinct emission spectrum when subjected to, for example, ultraviolet radiation is supplied. This may be a prescribed defined mixture of homogeneous populations of particles that have various levels of color centers of various types. The authentication in this case involves irradiation with ultraviolet light, and examining the spectrum or intensities, durations and wavelengths of emission and matching these with data supplied by the manufacturer. This may be done by having the user or purchaser obtain the spectrum or spectral data using a detector, supplied by the manufacturer or otherwise made available to the purchaser, to obtain the spectrum or emissions which can then be evaluated on site or electronically transmitted to a data center for verification—typically using a programmed interrogation device. Correlating a product identification number with spectral data and comparing the spectral data of the tested product to the data for the authentication system programmed into an interrogation device allows verification of authenticity and if done at a data center (based on electronically conveyed product spectral data) allows the data center to notify the user of the authenticity of the product. For example, a pharmacist purchasing an oral dosage form of a drug would expose the dosage form to a detector that obtains these spectral data and transmits them electronically to the data center. Alternatively, the detection function and interrogation function are integrated in the same device or apparatus, which may be programmed to use only certain excitation parameters and/or to detect only certain emission parameters.

In one embodiment, illustrated in the examples below, a preparation of diamond particles is separated into populations each of which emits a distinctive color, such as red, yellow, green or blue by any convenient method, such as flow cytometry. Irradiation with ultraviolet light of the appropriate wavelength will then effect emission of an individual color from each separate population. These populations in prescribed mixtures can be applied to products and their presence detected with the naked eye, as well as by precise spectra. By varying the patterns or ratios of the individual colors, various authentication codes one for each prescribed form of the authentication system will result. For example, both green particles and red particles could be applied in one case or green particles and blue particles in another, or simply red or simply green or simply blue in various proportions. It is sometimes helpful to have the various populations arranged in a pattern on a surface of the substrate so that the variation in the pattern is also distinctive, although in some cases overlap permits distinction—e.g., yellow and blue appears green. Differing intensities could also be employed as distinguishing feature, although if the naked eye is relied upon to distinguish intensities, the number of intensity levels available may be relatively small. Nevertheless, a wide number of authentication patterns can be employed using various combinations of these populations as individual prescribed forms of the authentication system.

Alternatively, a system that permits the purchaser to identify the product on the site of purchase or use involves matching the excitation wavelengths, intensities and durations to the emission wavelengths, intensities and durations according to a code included in the packaging or otherwise associated with the product.

In this more complex form, the code would inform the purchaser of the correct intensity and duration of the excitation wavelength such as that provided in Table 1 and the expected observed color, which would be visible at its relevant intensity to the naked eye. This could be done using a single population of particles, or a set of two or more homogeneous populations thus permitting a wider variety of fingerprints that could be discernible by the purchaser. This embodiment also may employ identification and verification by a data center after transmission of the spectrum or spectral data of the product which putatively contains the prescribed form of the authentication system to an interrogation device in the data center. The interrogation device could be a computer programmed to compare authentic spectral data to the data received. As described below, by varying not only the emission wavelengths, but employing ZPL determination, and/or intensity and/or duration determinations, a large number of distinct fingerprints can be generated. The distinction, however, may not be immediately discernible by the naked eye, but would require determination of the emission maximum wavelengths or ZPL's and/or intensities and/or duration in a more complex manner, and comparison could be made by a handheld interrogation device that compares spectral data of the prescribed form to the product spectrum on site or at a data center.

The invention is also directed to compositions prepared by the invention method as well as to methods of authentication which involve irradiating a product to be authenticated with the appropriate excitation wavelength of appropriate intensity and duration to generate fluorescence and to observe the fluorescence. Typically, the energy of excitation is higher than that of the emitted wavelength although by using two photon excitations the sum of the photons represents the excitation energy and thus the wavelength of each photon in the excitation spectrum may be longer than the wavelength of the emitted energy. Typical spectral emission in the visible range results from irradiation with ultraviolet light, although visible—visible emission excitation is also known (see FIG. 17), as is two photon excitation from the IR to result in visible emission. The observation may be direct visual observation with the naked eye or may involve a complex spectrum generated by the appropriate excitation energies, and determined by a detector which may be a spectrophotometer and compared to an authentic spectrum by eye, or may employ a programmed detector that includes an interrogation device.

### BRIEF DESCRIPTION OF THE DRAWINGS

FIG. 1 shows a color photo of separated populations of red, green and blue diamond particles viewed under UV light.

FIG. 2 is a color photo of an oral dosage form to which red, green and blue diamond particles have been affixed as viewed under ultraviolet light.

FIG. 3 is a color photo of a blister pack of dosage forms to which diamond particles have been added and viewed under ultraviolet light.

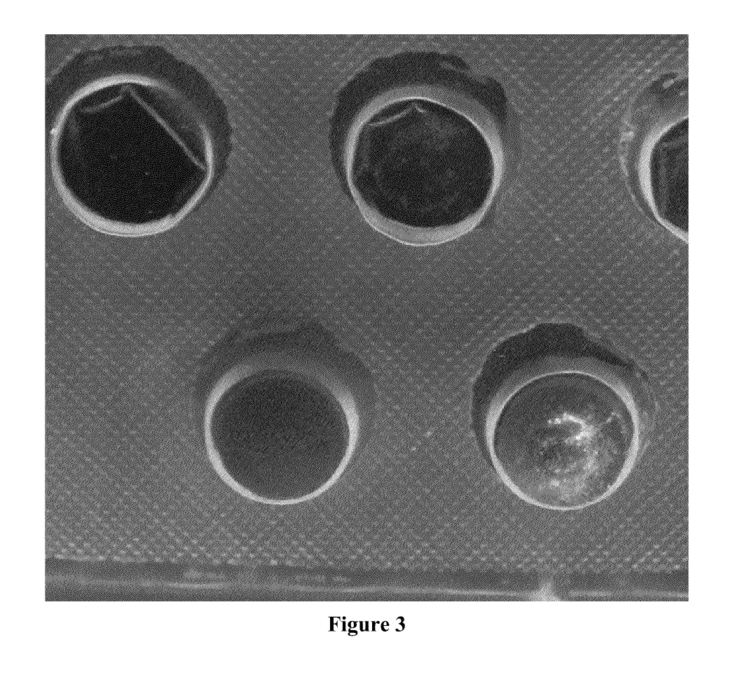

FIG. 4 is a color photo of cuvettes containing particle suspensions of red or green, or red-green mixtures or red/green/blue mixtures viewed under ultraviolet light.

FIG. 5 shows the visible emission spectra upon UV excitation of single color (red, green or blue) particles.

FIG. 6 shows the visible emission spectrum upon UV excitation of mixtures of these particles.

FIG. 7 shows solid dosage forms doped with red, green or blue particles.

FIG. 8 shows the IR emission spectrum upon excitation with visible light of unseparated mixtures of diamond nanoparticles integrated into a solid substrate.

FIG. 9 shows the visible emission spectrum obtained from such mixtures upon excitation with a wavelength of 365 nm.

FIG. 10 is a photograph of nine different tablets composed of standard pharmaceutical excipients which have been tagged with various fluorescence colors of diamond particles or mixtures thereof.

FIG. 11 shows a composite of spectra obtained individually from the tablets that contain red fluorescent particles only, green fluorescent particles only and blue fluorescent particles only.

FIG. 12 is the emission spectrum between 400 and 700 nm of tablets that contain mostly red fluorescent particles but also a trace of green and blue.

FIG. 13 is an expanded depiction of the portion of the spectrum in FIG. 12 between 400 and 550 nm.

FIG. 14 is a composite showing the spectrum of each of the nine tablets shown in FIG. 10 over the 400-700 nm range.

FIG. 15 shows the integrated forms of either the entire emission range in terms of total intensity counts or over the individual peaks defined by the individual components.

FIG. 16 shows one embodiment of a system for authenticating and verification of authentication of products through stored data and algorithms.

FIG. 17 shows a visiblevisible spectrum where excitation light is blue and emission is red.

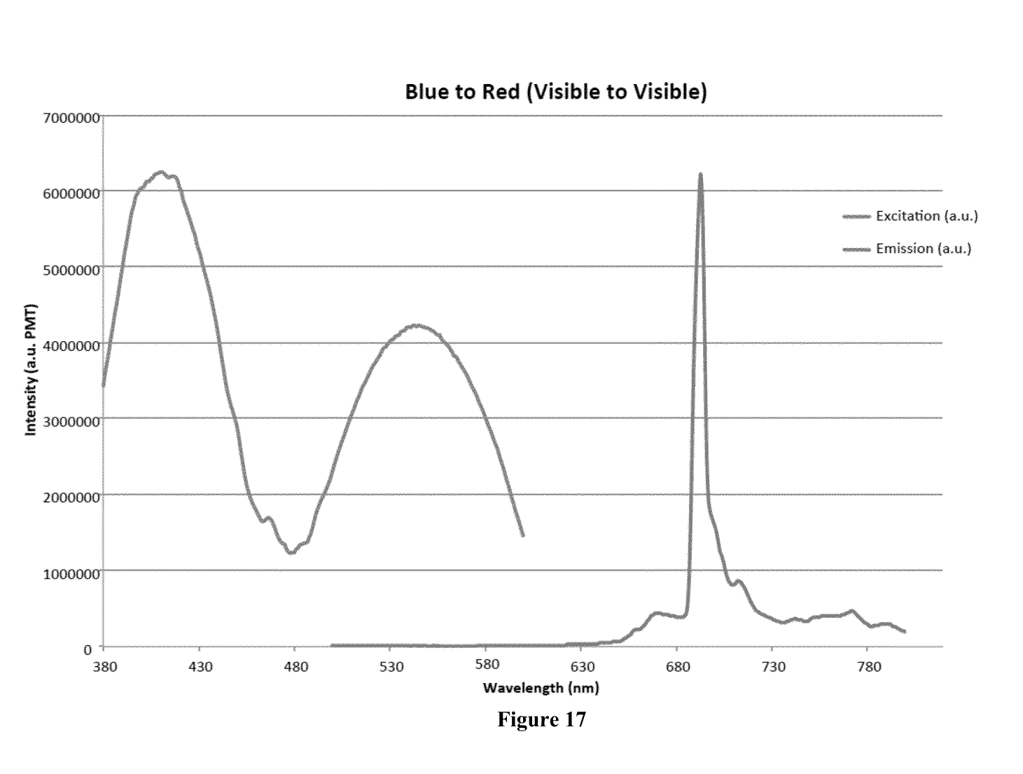

### MODES OF CARRYING OUT THE INVENTION

The invention provides an authentication system for a wide variety of products including pharmaceuticals, paints, oils, textiles, currency, food, and a multiplicity of other products that can be formulated to include diamond particles. For many applications, it may be useful to employ microparticles or nanoparticles. “Microparticles” means particles of diamond that have average diameters in the range of 1 μ to 1 mm, more typically 1 μ to 100 μ. “Nanoparticles” refers to diamond particles that have diameters between 1 nm and 1,000 nm, typically in the range of 10 nm-500 nm or 10 nm-100 nm. In some applications, a particular size of particles may be preferred. Microparticles, for example, may be appropriate for orally administered compositions. Particles in the micron range have been shown to fluoresce, perhaps more brightly than those in the nanometer range, by, for example, Bradac, et al., Nano Lett. (2009) 9:3555-3564; Boudou, J.-P., et al., Nanotech. (2009) 20:235602.

The size of the particles useful will depend on the particular application. For example, in the context of currently available printing equipment, typically, particles should be no larger than 5 microns. For use in pharmaceutical tablets, for example, a typical size might be approximately 100 nm. There is no hard and fast rule, however, and these are merely suggested sizes. It will be apparent to the practitioner for a particular application what range of sizes is suitable.

Also important to the invention is the definition of specific “populations” of diamond particles. The population may be heterogeneous or homogeneous. By a homogeneous population is meant a collection of particles that all have the same excitation and emission spectrum. By the same spectrum is meant that the location of the excitation and emission wavelengths and the intensity and duration of emission based on a particular intensity and duration of excitation is the same for all members of the population within a range sufficiently small that the population is discernible as a distinct population. The level of homogeneity will depend on the manner in which the populations are to be used. For example, if all that is necessary is to separate the particles into populations of different colors that are distinguishable by the naked eye, the level of homogeneity with regard to intensity may not be relevant. All that is necessary is to provide a population that is sufficiently homogeneous to be seen as red, or a population that is sufficiently homogeneous to be seen as yellow or green or blue as the case may be. On the other hand, if the authentication requires the generation of complex levels of detection which require particular intensities or specific wavelengths of emission, the populations may need to reach higher levels of homogeneity, possibly as high as that wherein at least 90-99% of the particles in the population have the same absorption maximum and possibly do not vary in intensity by more than 1 or 2%. Depending on usage, the variability may be greater.

The “prescribed form” of the authentication system refers to the particular population or mixtures of populations of diamond particles that are used in a particular authentication system with respect to a particular product. The product to be analyzed will either have the prescribed form contained within it, in which case it is indeed authentic, or it will have no authentication system or a different authentication system in which case it is not authentic. The product or packaging to be tested will be tested for this prescribed form, and it may or may not in fact contain it.

The prescribed form is typically designed by the manufacturer or by a supplier and under the control of the designer. Because the authentication systems consist entirely of inert diamond material regardless of the proportions of any of the various populations in the prescribed form, the designer is at liberty to select from a multitude of possible variations.

As used herein, “product” or “substrate” refers to the material which is to be authenticated. Thus, whether the product or substrate is a tablet, a piece of cloth, a solid article, a powder or a liquid composition, an emulsion or a semisolid, an appropriate authentication method employing the diamond particles of the invention can be designed. “Product” also includes packaging, as well as intermediates which are to be converted to product. For example, if the product is a finished pharmaceutical dosage, the active pharmaceutical ingredient (API) may be labeled. Any intermediate that is carried over to the final product can be labeled.

The authentication of the labeled product involves detecting spectral data from a tested product and comparing these data the corresponding data in the authentication system for that product. The determination of these data and the comparison may be performed simultaneously in the same apparatus or separately in the same apparatus or in two different instruments that may be in the same or different locations. Thus an apparatus may be programmed to interact with the product based on predetermined parameters and register a match or no match. The components which interact with the product for spectral data determination and which make the comparison may thus be the same or different in the same apparatus. However, these functions may be entirely separate and done by two different instruments and the different instruments may or may not be at the same physical site, since the spectral data can be transmitted, optionally in encrypted form, to an interrogation device at a remote location.

While very important products as subjects for the authentication method of the invention are pharmaceutical compositions, including those for oral administration as well as alternative formulations such as biologicals or parenteral formulations, a wide variety of products can be authenticated using this labeling system. This is important, for example, in connection with luxury goods where verification of point of origin is critical to prevent piracy. Illustrative goods include cosmetics, fragrances, clothing, accessories such as wallets or purses, and the like. Inclusion in ink used to identify the packaging of goods as trademarked is also important and both the trademark itself and the trademarked product can be similarly labeled or labeled with different compositions of the invention. Other important substrates include documents, currency, inks in general, and any product where either the origin of manufacture or other index of authenticity is important. In some instances, especially where there is a known problem of undercutting regulated products so as to undermine their safety, authenticating products such as foods by the invention method is a solution to the problem. Textiles, paints, mechanical parts and documents of value, such as stock certificates and monetary instruments may be labeled according to the invention.

One particularly useful embodiment relates to “solid oral dosage forms” or SODF's for which the FDA has issued guidelines for authentication using physical-chemical identifiers. SODF's include without limitation, tablets, capsules containing powders, gels and the like.

As used herein, articles such as “a”, “an” and the like are generally used to mean either one or more than one unless otherwise indicated. Further, where ranges of parameters are disclosed, where the ranges include integers, all integers within the cited range are included as if specifically set forth. For example, a range of variation of 4-10 possible intensities would specifically include variations that include 5, 6, 7, 8 or 9 different intensities. This stipulation is in order to avoid repetitious explicit enumeration and make the present specification more readable.

The homogeneity of individual populations can be assured by preparing diamond particles according to methods known in the art that generate specific color centers that are associated with particular spectra by controlling the conditions so as to result in a homogeneous population. The number of such color centers will determine the intensity of fluorescence. The homogeneity of the populations can also be assured by separating mixtures of diamond particles into homogeneous groups, for example, by flow cytometry. It has been shown that commercially available diamond particles can indeed be separated into individual color populations by this method. Thus, populations that are sufficiently homogeneous for a particular method can be obtained using standard techniques.

Homogeneous populations are particularly useful in preparing controlled authentication systems where visible color is used by the purchaser to authenticate the product on site by using a specific excitation wavelength and observing a defined color. For these systems, it is typical to use a combination of at least two populations, or more—three populations, four populations, five populations, etc., depending on the number of colors that the user is asked to observe. If only a single color is to be observed, then the particles may be distributed throughout the product, for example, if the product is a foodstuff or a pill, the user can be instructed to employ a particular excitation wavelength and instructed to expect to see, for example, red or yellow or blue. However, if a combination of colors is expected, it may be desirable to distribute the particles in a pattern on the surface so that the particles that emit, for example, green, can be readily distinguished from those that emit red. This is not always necessary since more than one color seen together will provide a different hue—e.g., red and blue looks purple. These combinations may also take advantage of differing intensities and/or durations of emission in the populations, but this determination is generally more complex since determination of intensity and duration levels with the naked eye is difficult, especially discerning among a reasonably large number of such levels. It is contemplated that, for example, intensity levels differing over a range where 5 or 7 or 10 intensity levels could be specified, but would require detection devices. However, the user could verify his initial authentication by obtaining an emission profile of the authentication system and sending it electronically to a data center that is able to display and match the relevant spectral data with those of the product. Alternatively, this could be done on site using an appropriate interrogation device.

It should also be noted that visual appearance from a combination of homogeneous populations may not be intuitive. For example, as shown below, a 1:1:1 mixture of red:green:blue particles appears yellow.

Thus, for homogeneous populations, in one very simple embodiment, a single population of diamond particles may be used. To use a pharmaceutical dosage form as an example, the authenticity of the composition can be verified by the end-user by illuminating the formulation with the appropriate wavelength and discerning the presence or absence of the expected emission color simply by visual detection. A simple emission spectrum may be obtained using a spectrophotometer. If desired, this can also be authenticated by a more complex readout of the spectrum including, optionally, the identification of the zero phonon line (ZPL) which represents pure excitation absent variation due to alteration in vibrational states and by measuring duration. The existence of a ZPL is emblematic of diamond and its measurement can be used to confirm the presumed presence of this material.

For straightforward detection without any sophisticated measurement of spectra, a number of devices are readily available. As noted above, an LED light that emits 360 nm is commonly available and this is capable of excitation of emission in the visible range of varying colors depending on the nature of the particles themselves. In addition, for a modest cost, devices are available that permit different excitation wavelengths to be employed as displayed by the device and the corresponding emission wavelength(s) can be displayed numerically or would be visible to the naked eye.

Devices are available that also detect emissions in the infrared and can detect levels of intensity.

While the system described in the previous paragraphs is effective per se, it is advantageous to use a more complex authentication system in order to provide a specific authentication for a particular batch or a particular type of dosage. By employing more than one population with varying, for example, just the emission/excitation wavelength combinations and intensities, a very large number of distinctive patterns can be generated.

For example, using 4 colors and 10 intensity levels, many thousands of different patterns can be obtained. Adding duration of the emission as a variable results in even more possibilities.

If even more colors are used or more intensities are used, the number would be even higher. Thus, a large number of combinations can be prepared to distinguish individual batches or individual formulations. There would be a sufficient number of individual signatures thus, to permit the identification of individual batches, for example, of a pharmaceutical dosage not just to verify the nature of the drug itself. Even larger numbers of alternatives can be prepared by varying among more colors or including more different levels of duration and/or intensity.

In some embodiments, it is advantageous to use a heterogeneous population of particles so that complex emissions are obtained. Populations with random assortments of particles with varying numbers of color centers and varying types of colors centers can be obtained, and can occur in nature. These have inherently high flexibility. For a random heterogeneous population, unique emissions would be generated, by irradiation with light of sufficient wavelength to excite various color centers in the random mixture at various intensities and durations. This embodiment works best with respect to obtaining data on site which is electronically transmitted to a matching facility to permit authentication; however, if facilities or a programmable detector incorporating interrogation device are available, on-site determination may also be practical. Alternatively, the product could be sent off site for authentication.

A particularly useful combination is that of an unseparated mixture of diamond particles as a fraction of the total label where the remainder of the label consists of one or more homogeneous populations of the separated forms. The individual separated forms generate discrete emission peaks, while the unseparated mixture is relatively silent in terms of visible emission but has a characteristic infrared spectrum.

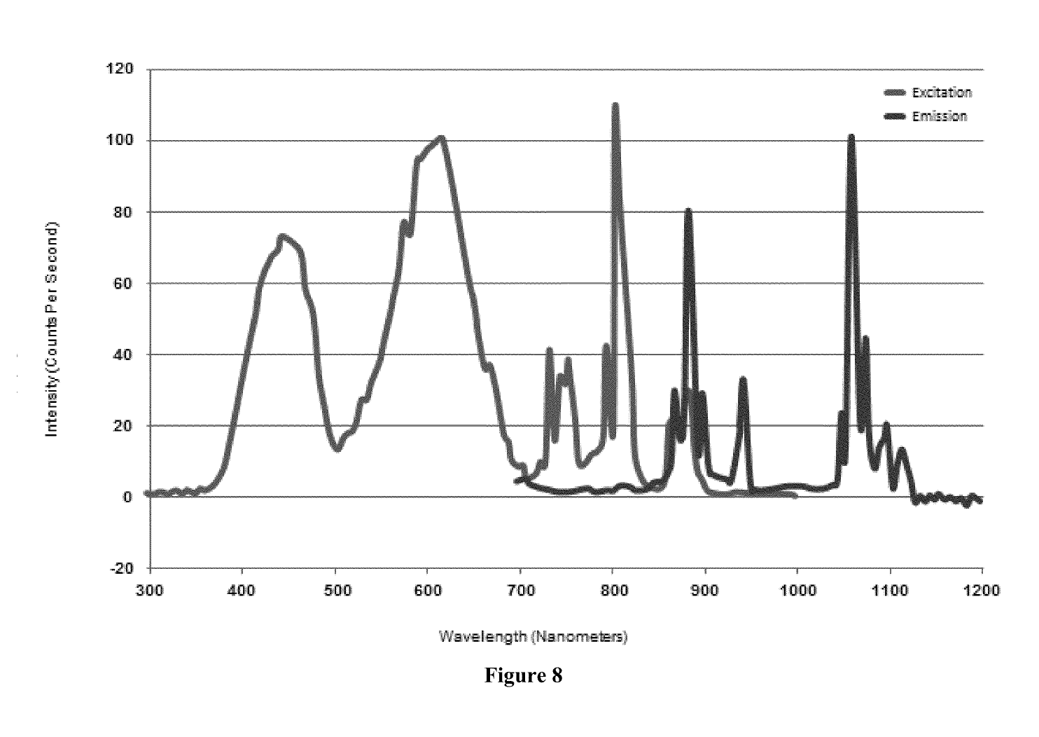

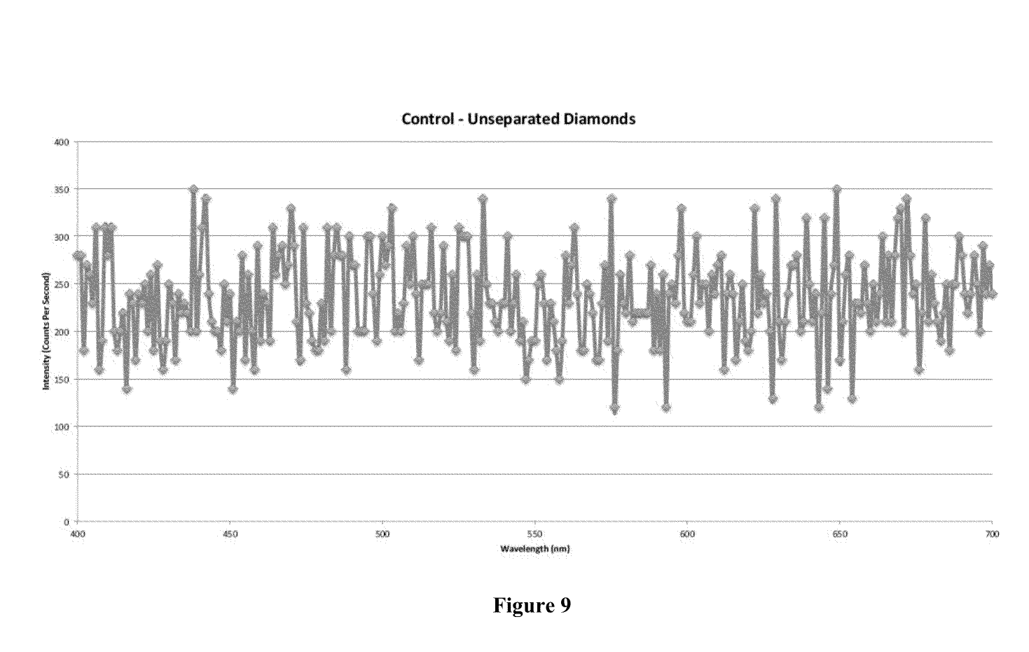

Thus, an extra level of authentication can be provided by adding to the known ratios of components a portion which constitutes unseparated diamonds. These mixtures appear black to the naked eye and also generate an essentially null spectrum as described in FIG. 9 in Example 5 below. However, as shown in FIG. 8, also in Example 5, this mixture provides a characteristic infrared spectrum that is excitable by visible light. This aspect of authentication is more difficult to counterfeit as mixtures, for example, of various dyes would not have this result. This unseparated mixture of diamond particles can be used alone or added as a portion of the label and superimposed upon the remaining separated components.

In that regard, by mixing various proportions of red, blue and green diamond particles, dosages or labeled substrates can be obtained that appear yellow to the naked eye but when examined spectroscopically clearly show the ratios of components. This is illustrated in Example 6 below. It appears that several dozen different spectroscopically distinguishable but visually indistinguishable yellow substrates may be obtained by varying proportions of these three components.

Another dimension of authentication can be obtained by adding to the substrate a hygroscopic organic component that becomes dehydrated upon application of pressure. This is particularly useful in the context of orally administered tablets because a particular hygroscopic organic material—magnesium stearate—is a common component of such dosage forms. This particular hygroscopic hydrophobic compound has the property of causing an indigo-violet shift in the spectrum known as a leafing effect. This leafing effect results also in a separation of diamond particles distributed between the magnesium stearate and the remainder of a hydrophilic substrate. When separated diamond particles according to color are included in substrates which contain the hygroscopic organic material and then subjected to pressure, for example, in making a tablet, the hygroscopic material is at least partially dehydrated resulting in what to the resident diamond particles appears to be a two-phase system. The substrate, for example tablets, then assumes a speckled appearance due to the uneven distribution of the diamond particles. This, too, is difficult to duplicate in a counterfeit material since typically only diamond particles exhibit this property of uneven distribution among the organic/hydrophobic, now dehydrated material and the remainder of more hydrophilic materials included in the substrate. Counterfeited substrates that substitute other fluorescent substances for diamond particles do not have this property.

The levels of particles required to result in successful detection depend to some extent on the method of measurement. It appears that to detect the presence of one or more colors of taggant visually, levels only of approximately 10-100 ppm, i.e., 0.001% -0.01% by weight, are required; or even 1 ppm or 0.0001% as a lower limit. However, very simple and commercially available instrumentation can easily detect 50-100 ppb. The lowest limit needed for detection depends on the sophistication of the detector and thus considerably lower levels could also be detected with the appropriate equipment.

Particularly where complex mixtures of diamond particles are employed, a more sophisticated system for identification is helpful. As noted above, a reasonably simple tagging method can be verified simply using a handheld LED device which permits visual inspection. Generation of a simple spectrum will also enable direct observation and evaluation of the spectrum itself, e.g., as printed out over a suitable wavelength range. On the other hand, especially but not necessarily where complex mixtures, rather than, for example, a particular design on the surface of a solid formulation are used, a programmed detector incorporating an interrogation device is often employed. Thus, the intensity and/or wavelength and/or duration of the various peaks or a selected portion thereof in the emission spectra of the particular combination of populations of diamond particles combined with the appropriate excitation parameters can be recorded in such a detector which can then either accept or reject authentication based on matching or non-matching of the embedded information with that generated by a physically obtained emission spectrum or portion thereof of the product or its labeling. These data may be assigned a code associated with the product which may be secret known to an authentication service provider.

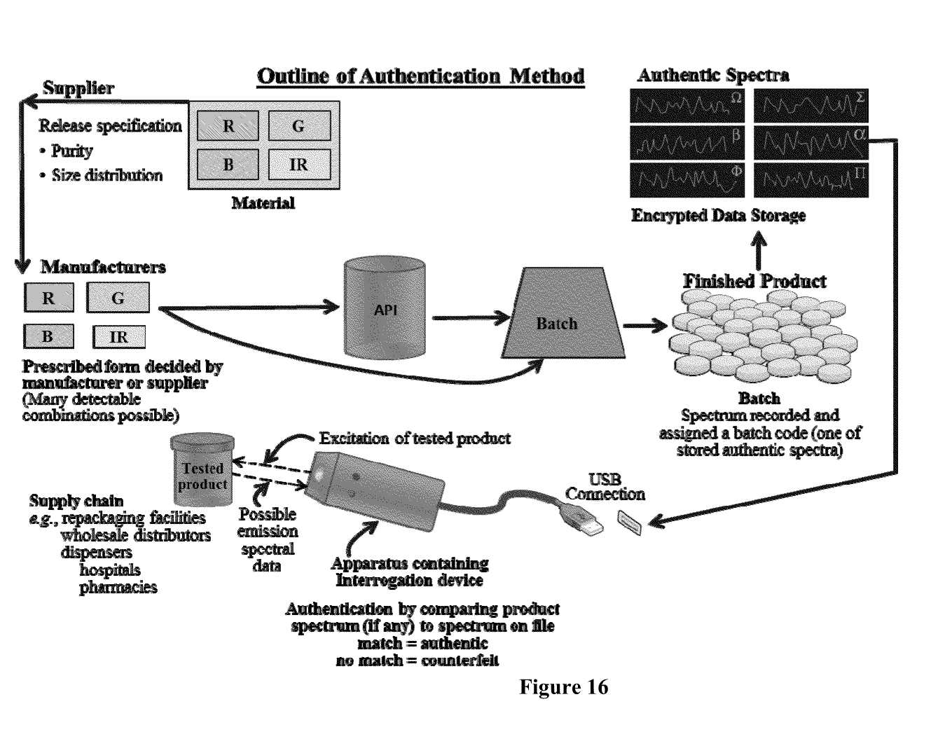

One illustrative but not limiting embodiment of the overall system as applied to an oral dosage form is shown in FIG. 16. In this exemplified procedure, a mixture of four populations of diamond particles is used—red (R), green (G), blue (B) and infrared (IR)—and mixed in various ratios. A particular mixture is illustrated in the figure. The composition of the mixture can be determined by the dosage manufacturer or a supplier. The mixture of specified proportions is then characterized in terms of its spectral characteristics and added either, in this case, to the active pharmaceutical ingredient (API) or to a batch used to prepare the finished product. In each case, spectral data are recorded from the API, batch or finished product and assigned a suitable code. It is preferable that the authentic spectral data for the product to be obtained from the product itself since the chemical and/or physical form of the product may influence these somewhat. Authentic spectral data from various products are encrypted for data storage and are programmed in advance into an interrogation device through a USB or other suitable connection. The interrogation device may be part of (as shown in FIG. 16) or may be separate from a detector for spectral data of the product to be tested in which case data from a detector are fed to the interrogation device. The interrogation device then attempts to match the authentic spectral data with spectral data obtained from the product(s) tested by comparing them. The detector shown in FIG. 16 (or the interrogation device in general) may be housed in a handheld apparatus, but need not be and may be remotely programmable, but need not be. (It is often more convenient to employ handheld remotely programmable devices, but this is not a necessity.) The match or non-match is then read—where there is a match, the tested product is considered authentic whereas if there is no match it is considered counterfeit.

Detectors of the type that can be configured to be programmable to match incoming spectra from programmed-in spectra are described in U.S. patent documents 2003/0173539; 2004/0169847; 2011/0090485 and 2013/0277576. In some cases, only predetermined spectral data are programmed for determination into the device.

The number of spectral parameters to be measured is dependent on the complexity built into the assay system. The possible parameters include the wavelength, intensity, and duration of the peaks in the excitation and emission spectra. However, it is not necessary in every case to measure each and every one of these parameters. It may be sufficient to measure only a subset, such as a combination of wavelength and intensity of the emission spectrum pattern holding the excitation energy constant. Alternatively, the excitation energy or intensity can be varied and a simpler form of the emission spectrum measured. The design of the levels of the various parameters available is well within the skill of the ordinary artisan familiar with the spectral patterns emitted by materials in general.

The system shown in FIG. 16 is only one of a number of possibilities. Various types of detectors can be used with various capabilities and the nature of the authenticating entity (e.g., the end user or a service provider) is variable depending on the design of the business arrangements associated with the technology.

In addition to tagging dosage forms or other products with the diamond particles, packaging for a product may be similarly tagged with the diamond particles corresponding to the taggant used in the product. Thus, an easy way to detect counterfeiting of the product would comprise labeling both the product and the packaging for the product with the same coded mixture of diamond particles wherein a discrepancy between the packaging and the product would indicate tampering. The packaging label is a useful substitute for package labeling that currently may embody a barcode. The necessity for the barcode is obviated by replacing it with the diamond particle taggant mixture included in the ink. The same prescribed form of diamond particles could be included both in the product itself and in the ink used to label the packaging. This is the most convenient arrangement, but clearly not the only possibility—each could be independently labeled and assessed accordingly.

All documents noted herein are incorporated by reference as if fully set forth.

The following examples are offered to illustrate but not to limit the invention.

### EXAMPLE 1

### Separation of Commercially Available Diamond Particles

Monocrystalline diamond particles were obtained from Sigma Aldrich. The product designation indicates the diameter of these particles to be in the micron range. The mixture was subjected to flow cytometry to obtain individual populations that are red, green or blue when exposed to UV light as follows:

One (1.0) gram of the monocrystalline synthetic diamond particles was pumped at a flow rate of 0.5 mL/min through a fluorescence spectrometer (LS-555, Perkin-Elmer, Co.) using a standard flow cell. The spectrum was measured at three different wavelengths corresponding to 410 nm (blue), 550 nm (green), and 675 nm (red) with a 10 nm bandwidth separation setting. The excitation slits were set to 5.0 nm and the emission slits were set to 10.0 nm. Material was continuously set to flow at a fixed rate.

After excitation from an Xe lamp through a single monochromator set to 363 nm and collimated, the light was passed through a polarizing filter then through the sample. Collection of each particle was performed mechanically after detection by a standard 950 PMT (Hamamatsu Co.) after separation by the emission monochromator. The desired population of red, green, and blue materials was collected by deflecting the particles out of the main stream by a piezo-electric device using a fluidic valve operating on one arm of a Y-shaped flow channel. The other channel collected the red material as well as any non-fluorescent materials. The final collection was 200 mg of blue, 350 mg of green and 450.0 mg of red material, representing 100% recovery.

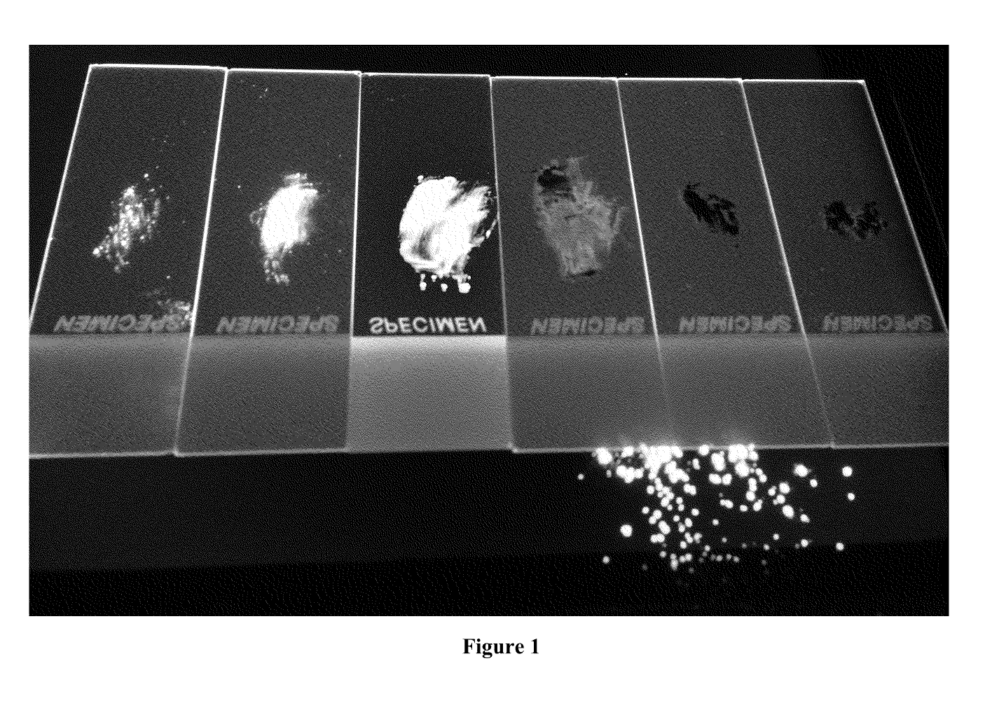

The visible emissions of the separated red, green and blue particles when subjected to UV light are shown in FIG. 1, slides 1-3. (Slides 4-6 are unseparated monocrystalline diamond from Sigma Aldrich, unseparated polycrystalline diamond from Sigma Aldrich and unseparated polycrystalline diamond from Mallinckrodt.) The loose bright material shown below the slides are particles of rare earth oxides YPV-F, from United Mineral Corporation, which is used a taggant, e.g., for currency or other documents, but is not used in pharmaceutical products.

### EXAMPLE 2

### Simple Labeling of Solid Dosage Forms

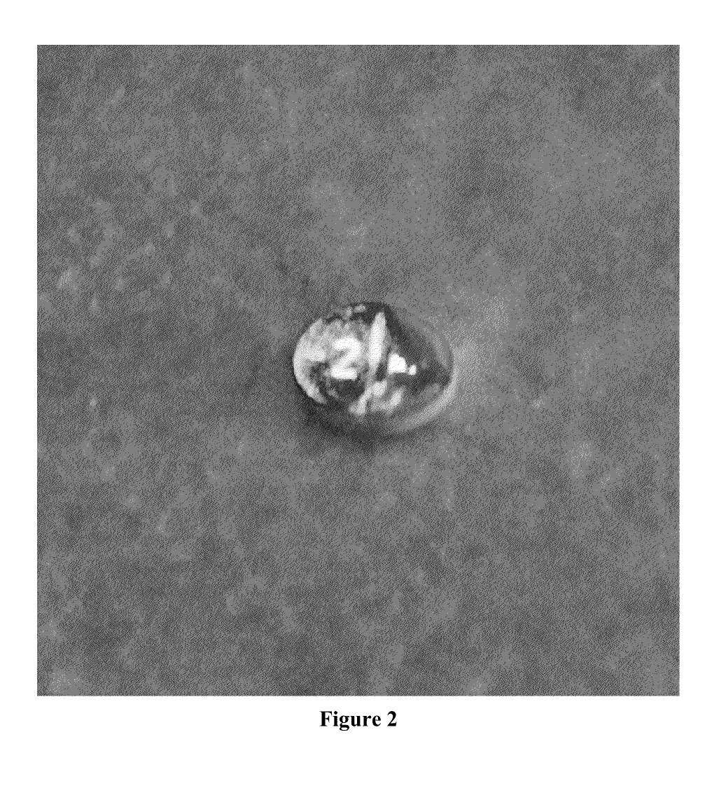

Using the separated particles prepared in Example 1, commercially available solid dosage forms were labeled with the individual populations by applying the separated particles with a Q-tip. FIG. 2 shows a photograph of the results of this straightforward application of the diamond particles to the surface of a tablet when irradiated with UV light of 363 nm. Red, green and blue fluorescence is seen. When exposed only to visible light, no color was visualized.

The red and blue populations prepared in Example 1 were also used to label commercially available tablets comprising similar fillers in a blister pack. As shown when exposed to UV light, red and blue populations are distinguishable through the blister. When exposed only to visible light, no color was visible.

### EXAMPLE 3

### Visual Appearance in Suspension

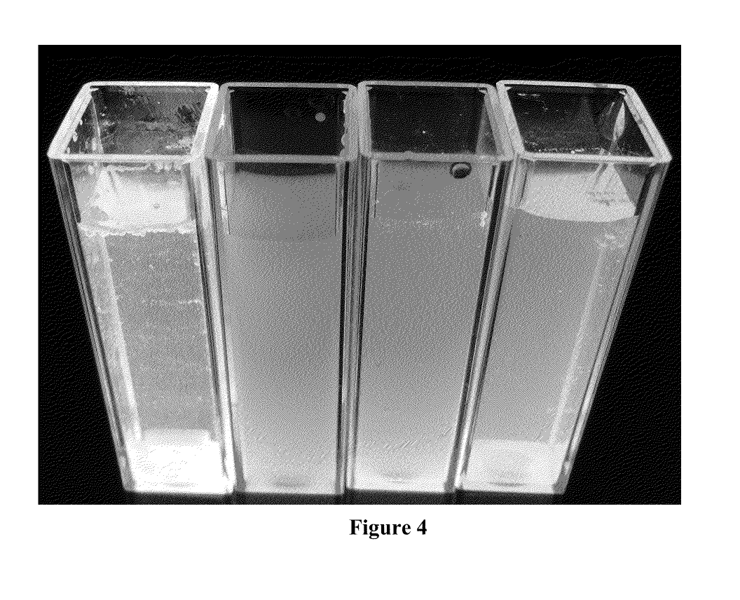

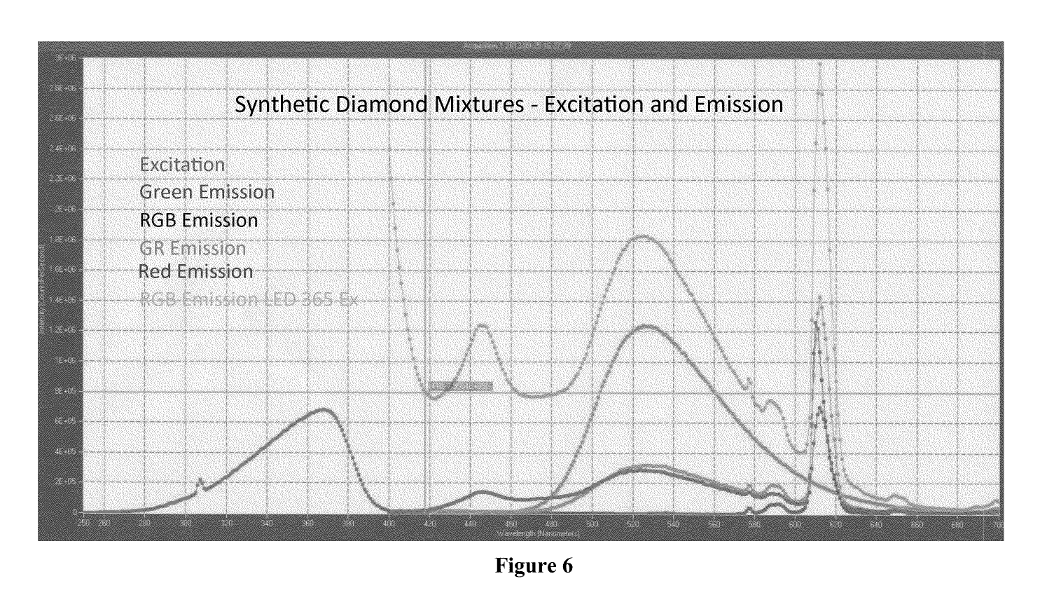

As shown in FIG. 4, the particles were resuspended in water in cuvettes. From left to right, these contain the green only, red only, red:green (approximately 1:5), and red:green:blue (approximately 1:4:2), all at 10 mg/ml. While the red and red:green (1:5) material appear to be the same, their spectral signatures are easily distinguished as shown in FIG. 6 (see Example 4).

### EXAMPLE 4

### Emission Spectra of Components

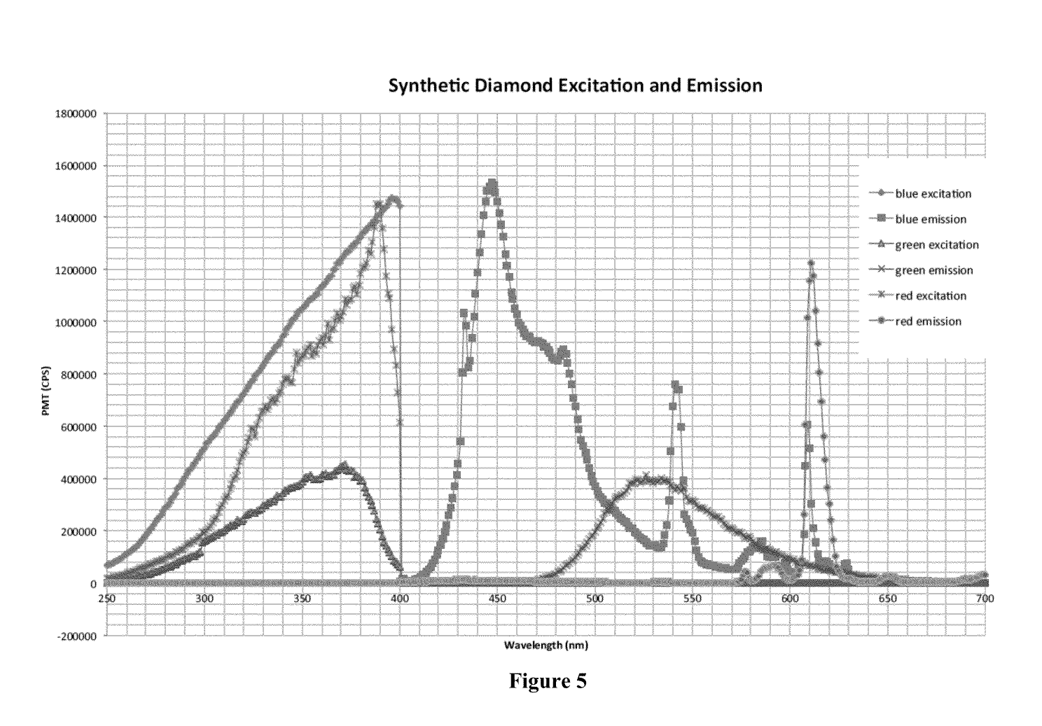

Fifty (50.0) mg of separated red, green and blue particles were suspended in purified water and placed into 1.0 cm square quartz cuvettes. The cuvettes were placed into a Photon Counting Machine (PTI, Inc.). The measurements were taken using a double excitation and double emission monochromators and a 400 nm long pass filter on the emission monochromator. Both mono gratings were 600 lines/cm with a blaze angle of 1.0 micron. Detection was achieved using a 950 p photomultiplier tube (Hamamatsu Co.). Excitation measurements were taken using an excitation algorithm and setting the emission monochromator to the maximum of each material and scanning the excitation from 300 nm to 450 nm, with results shown in FIG. 5.

The excitation wavelength for material fluorescing at all three colors was similar. The blue emission maximum was about 445 nm, the other peaks were likely due to green and red contamination. The emission spectra for green and red fluorescing materials appeared not to be contaminated by material that fluoresced at other wavelengths. The red emission spectrum contained some characteristic fine detail at 575 nm and 590 nm

Further measurements were taken in a similar manner using mixtures of the previously separated materials. It can be seen that each material that was mixed retains its unique spectrum in the visible range as shown by the emission spectrum in FIG. 6.

These spectra were obtained by setting the excitation monochromator in the spectrophotometer at 360 nm. An additional (fifth) spectrum arising from the red:green:blue fluorescing mixture, also shown in FIG. 6, was obtained by exciting the material, instead, with light from a hand held LED source at 365 nm. This spectrum appeared more intense because the LED source, as opposed to light from the excitation monochromator, flooded the sample chamber. Mixtures of these materials, surprisingly, retained, in the visible range, the characteristic spectral signatures of each component of the mixture. Rather than a single broad emission spectrum, one can clearly distinguish separate red, green and blue emissions in the red:green:blue mixture and red and green emissions in the red green mixture.

### EXAMPLE 5

### Visibility in Dosage Forms

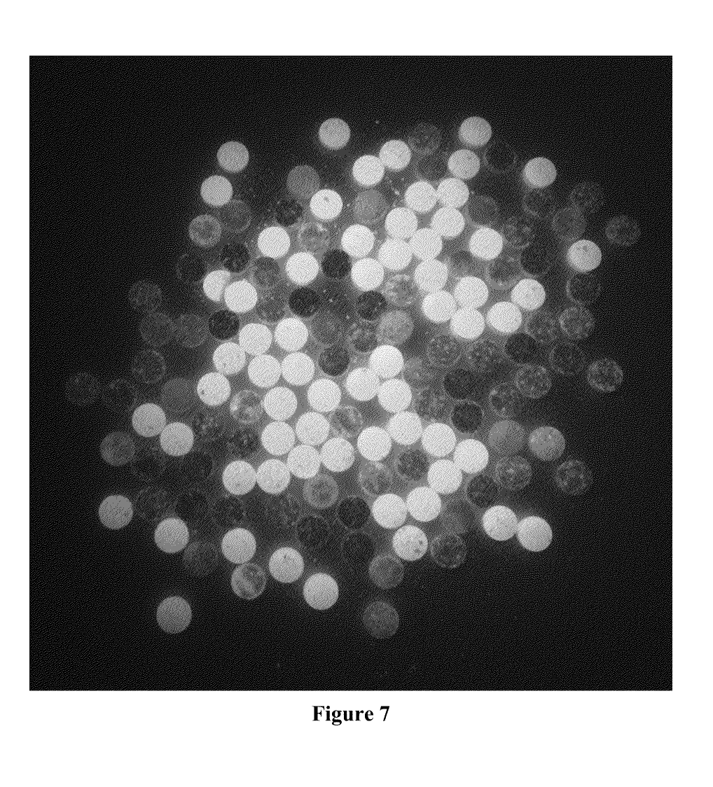

FIG. 7 is a photograph, taken under UV light, of pills comprised of calcium carbonate, hydroxypropyl cellulose and Avicel™ and approximately 1 mg/10,000 mg of red, green or blue material. This works out to about 0.1 mg or 100 micrograms of particles per pill. As seen, these fluoresce in various colors; while under visible light these pills appear identical. Pills that do not contain taggant or contain taggant that does not appear to fluoresce in the visible range appear black. However, tablets that contain unseparated mixtures of diamond particles appear black but are easily detectable by infrared fluorescence in the range of 850-1,200 nm, enabling forensic encryption.

FIG. 8 shows both the emission and excitation spectra of a tablet which contains a mixture of synthetic diamond particles at 100 ppm. Due to the interference of fluorescence from the various types of particles (e.g., red, blue or green), the tablets appear black and a spectrum obtained by irradiation with UV light at 365 nm in the 400 nm-700 nm range is essentially null as shown in FIG. 9. However, as shown in FIG. 8, there are characteristic peaks in the range of 850-1,120 nm in the infrared range which can be displayed when irradiated with light in the visible range, in particular in the range of 400-500 nm, 500-650 nm and 800 nm. A particularly strong peak at 880 nm is essentially an artifact of the spectrometer since the “emission” also includes reflected excitation light. The intensity of each peak in the IR range will depend on the excitation wavelength chosen and its intensity.

### EXAMPLE 6

### Comparison of Variously Labeled Tablets

Tablets comprised of standard fillers were prepared containing 100 ppm of various separated diamond particles or, as a control, unseparated mixtures of fluorescent diamond particles. As described in Example 1, the diamond mixtures were obtained from Sigma Aldrich and separated into red, green and blue fluorescence using flow cytometry. Eight different test tablets were prepared in addition to a control tablet which contains 100 ppm of unseparated diamond. The tablets were glued with transparent glue to a slide that has been painted black for better viewing.

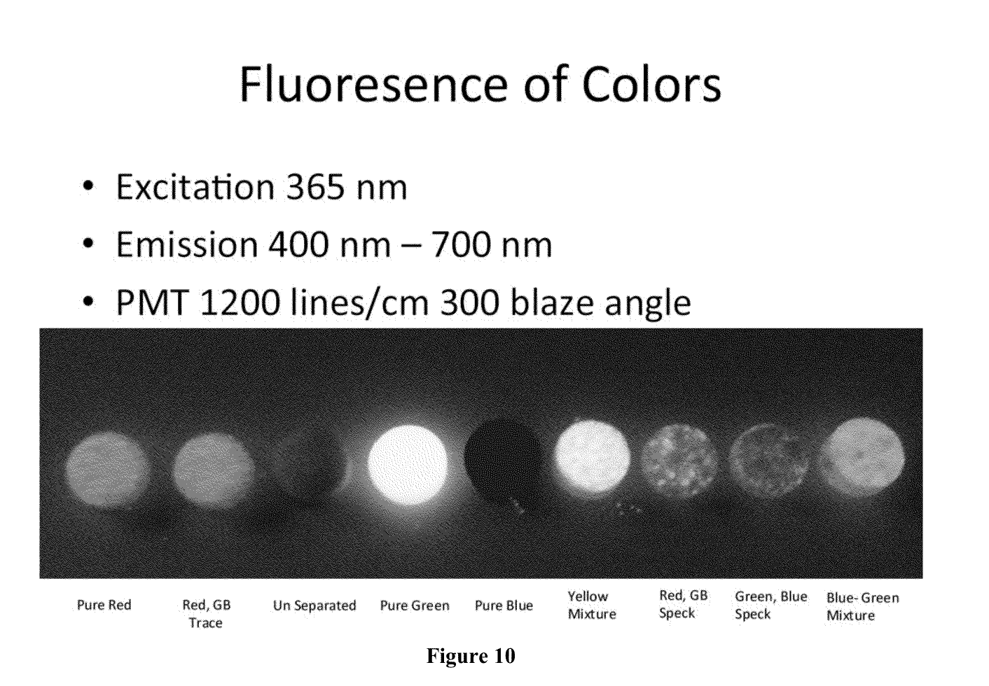

All of the tablets (except those labeled “speck” in FIG. 10) were prepared as follows. For those labeled “speck,” magnesium stearate was used instead of stearic acid.

| | | | Corresponding |
|---|---|---|---|
| Ingredient | Wt (%) | Supplier | Mass (g) |
| HPMC (hydroxypropyl | 69.9990 | Dow Chemical | 0.69999 |
| methylcellulose) | | | |
| Paracetamol | 13.2000 | BASF | 0.132 |
| Calcium Carbonate | 5.0000 | Dow Chemical | 0.05 |
| Ludipress ® | 3.0000 | BASF | 0.03 |
| Kollidon ® CL | 3.0000 | BASF | 0.03 |
| PEG 6k | 5.0000 | Hexion | 0.05 |
| Stearic Acid | 0.8000 | Sigma Aldrich | 0.008 |
| Diamond particles | 0.0010 | Persis Science, LLC | 0.00001 |
| Total: | 100.0000 | | 1. |

All components were mixed in a speed-mixer, sieved through 325 mesh screen and pressed with low compression. Total weight per tablet 680 mg, 13 mm diameter, and diplanar in form.

FIG. 10 shows the visible colors resulting from excitation at 365 nm. From left to right, the first tablet contains only red-fluorescing diamond particles, the second contains red-fluorescing particles with a trace of green and blue particles, the third contains an unseparated mixture of the original diamond particles before exposure to flow cytometry to separate colors, the fourth is a tablet tagged with only green-fluorescing particles, the fifth is a tablet tagged with only blue-fluorescing particles, the sixth is a tablet which contains equal amounts of red-, green- and blue-fluorescing particles and appears yellow, the seventh is a tablet that contains red-fluorescing particles with a trace of green and blue and also contains magnesium stearate as a component of the tablet itself, the eighth is a similar tablet containing magnesium stearate with an equal mixture of green- and blue-fluorescing particles and the ninth is a tablet also with an equal mixture of green- and blue-fluorescing particles but with stearic acid rather than magnesium stearate. The total level of diamond nanoparticles in all nine tablets is 100 ppm.

In all the following spectra, the y-axis measures the intensity in counts per second in the spectrophotometer.

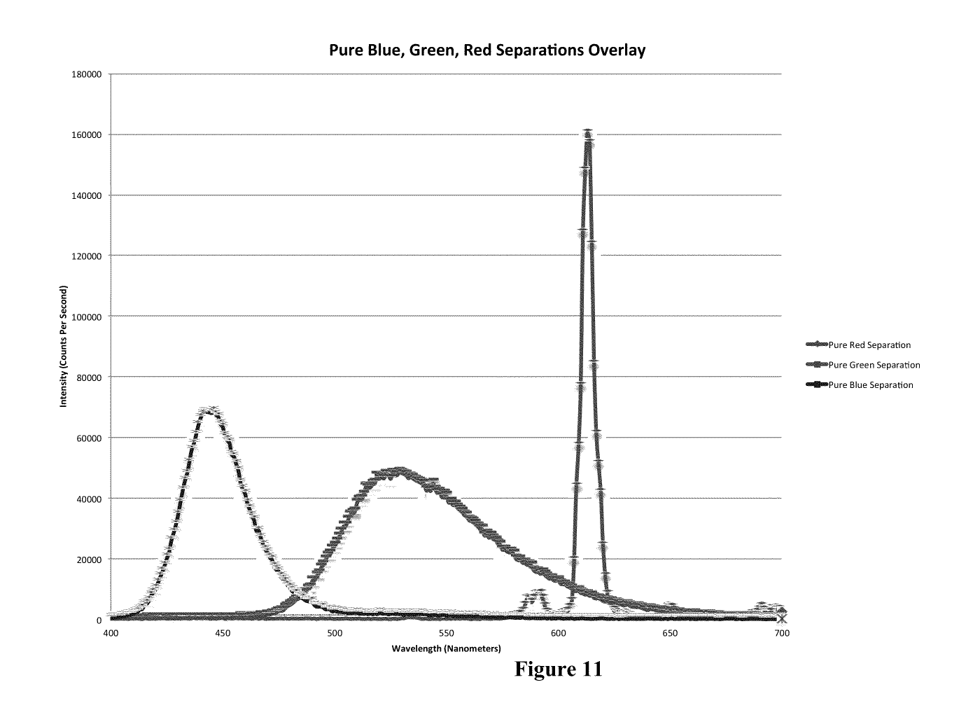

The individual spectra of the red-labeled tablets, the green-labeled tablets and the blue-labeled tablets are shown superimposed on FIG. 11. These spectra were obtained using a photomultiplier tube with 1,200 lines/cm and a 300 blaze angle. The emission was read at an angle of 44° from the excitation beam.

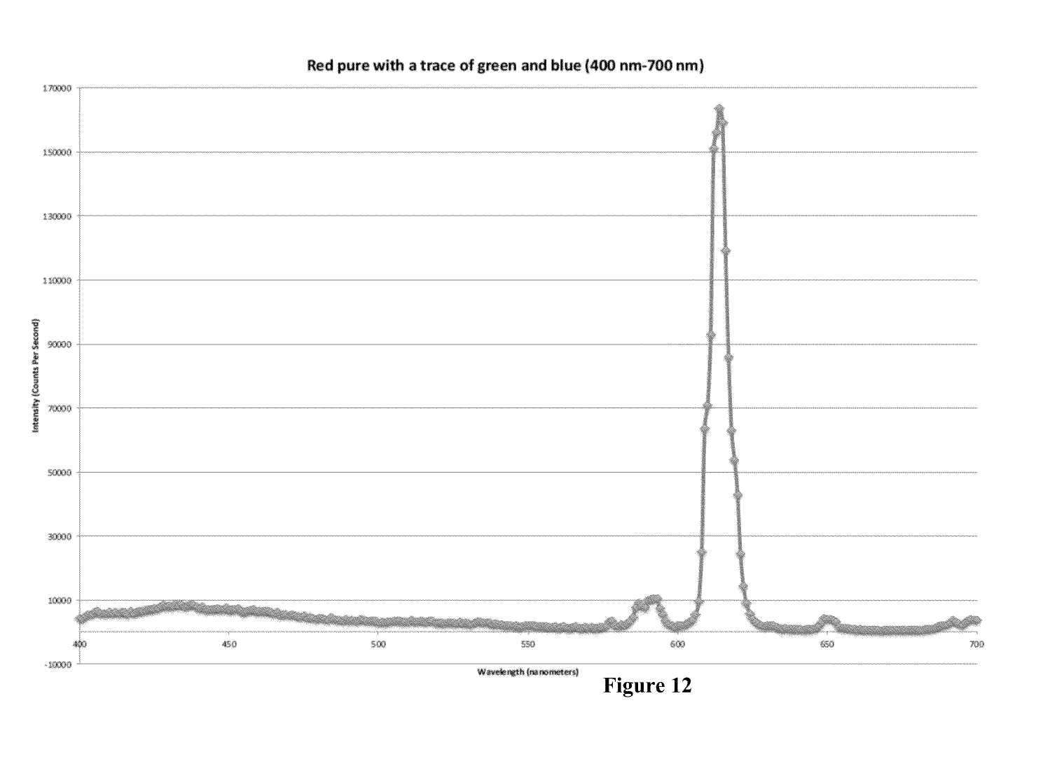

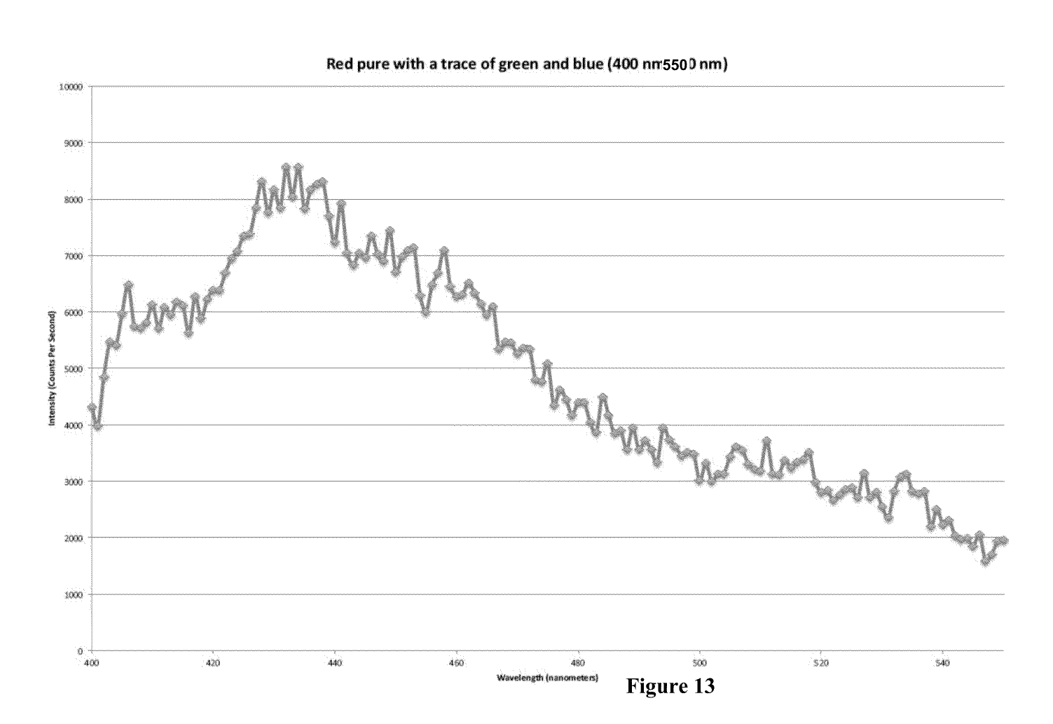

FIG. 12 shows the spectrum obtained in the same way for the second tablet from the left which contains red with a trace of green and blue. This is expanded in the range of 400-550 nm in FIG. 13 so that the contribution of the green and blue portions of the spectrum can be more accurately determined.

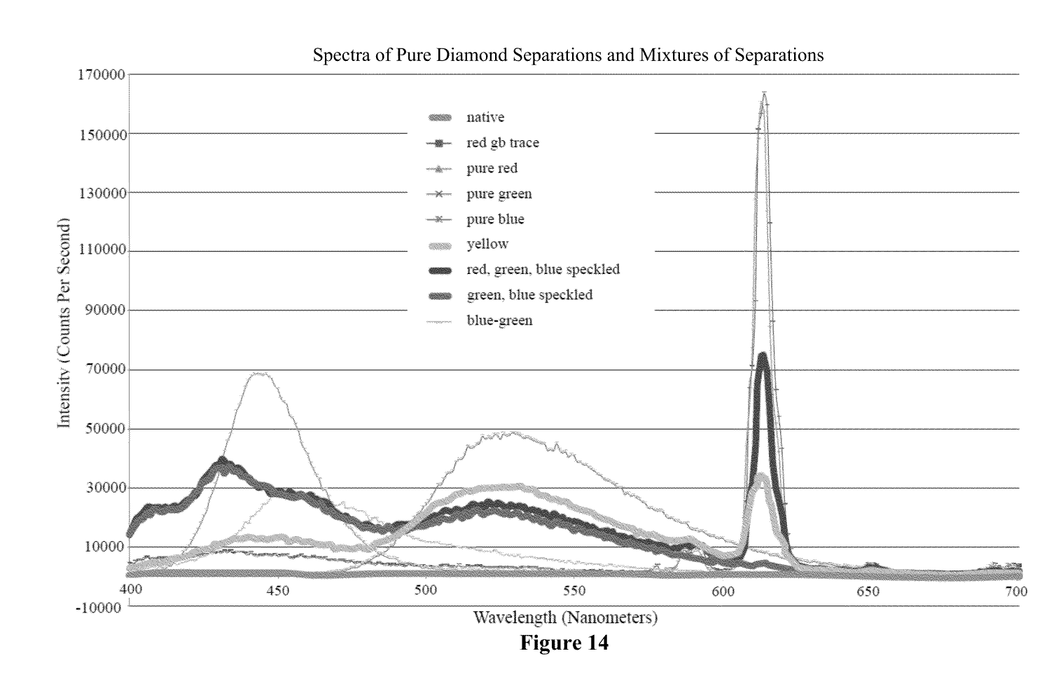

FIG. 14 shows superimposed spectra in the 400-700 nm range for all of the nine tablets depicted in FIG. 10. Of particular interest is the spectrum of the yellow tablets which shows distinct peaks in the red wavelength, the green wavelength and blue wavelength. The intensities of these are similar to those depicted in FIG. 11, except that the intensity of the blue portion of the spectrum appears more widely distributed over the wavelength band.

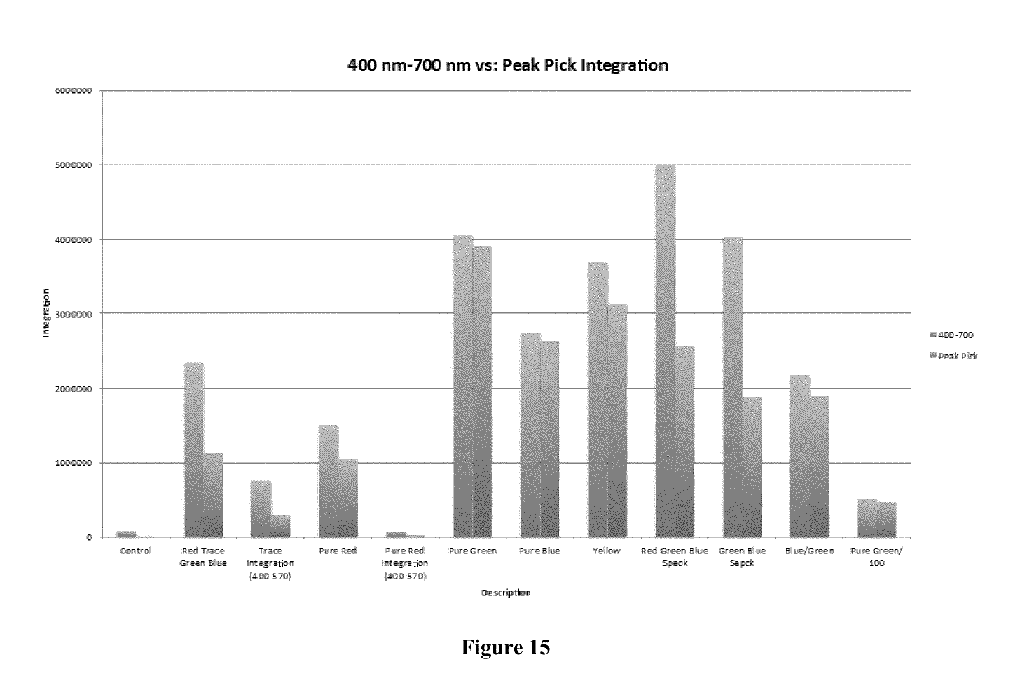

The data shown in these figures is compiled in FIG. 15 which integrates the number of photons over the entire spectrum (shown in blue) or over the relevant peaks (shown in red). In all cases, the red peak values will be smaller because they cover only the relevant range rather than the entire spectrum.

Reading from left to right in FIG. 15, the control is integrated over the entire 400-700 nm regions and is very low. A comparison between the range (400-700 nm) integration with the peak pick integration shows the specificity of the emitted wavelength. Thus, for the pure red-labeled tablets shown in the fourth set of bars, the integration over the entire range shows that the red peak accounts for most of the total intensity and the integration over 400-570 nm which excludes the red peak is minimal. Similarly, the pure green and pure blue labeled tablets, shown in the succeeding fifth and sixth comparisons, shows that most of the integration over the entire range is due to the individual green or blue peak. Turning back to the second and third from left comparisons where a red labeled tablet has a trace of green and blue, the integration over the red peak again offers a substantial portion of the overall integrated count, and the trace blue and green component relatively little (400-570 nm).

With respect to the results for yellow, each of the red, blue and green peaks were summed to obtain the peak pick integration shown in red and the intensity over the entire range is very little different is shown in blue. With regard to the tablets that contain magnesium stearate which show a speckled appearance, it is clear that the overall spectrum is much less defined in terms of individual peaks by comparing the red and blue bars. The concentration of colors in the blue and green peaks is shown in the tenth set of bars and the same tablet but with the intensities divided by 10 is shown in the last set of bars. This shows that 10 ppm could readily be determined on the equipment employed. Clearly, the lower level of detection will depend on the design of the spectrophotometer and the settings used.

### SUMMARY

The invention provides the following embodiments: (In all cases below “product” also includes packaging and intermediates.)

The invention provides a method for providing authentication to a product which method comprises combining said product with a prescribed form of an authentication system which comprises at least one population of diamond particles wherein said particles exhibit fluorescence, and wherein the wavelength, duration and intensity of the fluorescence emission of said particles is dependent on the wavelength, duration and intensity of the excitation energy; in some embodiments the population is homogeneous.

The method also includes an embodiment wherein said combining is with at least two homogeneous populations of said particles, wherein the fluorescence wavelength, intensity, duration or any combination is unique to each said different population.

In addition the method includes an embodiment wherein, in addition to at least one homogeneous population(s), the product is combined with a heterogeneous population of diamond particles, or the product may be combined only with a heterogeneous population.

In all of these cases, the populations are optionally distributed within the product, or the product may be a solid having a surface and the populations are disposed on the surface of the solid. If the latter, the populations may be disposed in a predetermined pattern on said surface.

The invention further includes a product prepared by any of the above methods. The product may be a pharmaceutical product, and may be in solid oral dosage form.

The above product may optionally be associated with a code designating the excitation wavelength(s) and/or duration(s) and/or intensity(ies) that cause said population(s) to fluoresce and/or identifies the emission wavelength(s) and/or duration(s) and/or intensity(ies), which code may be secret.

The invention also includes a method to authenticate a product to be tested which method comprises irradiating the product with excitation wavelength(s), duration(s) and intensity(ies) that generate(s) fluorescence from said population(s) of diamond particles and observing said fluorescence. In one typical embodiment a spectrum comprising both wavelength and intensity from each population may be observed.

In the above authentication methods, said test product spectrum may be evaluated visually or by use of a spectrophotometer or by use of a detector programed to consider only predetermined spectral parameters including a detector comprising an interrogation device either on site or spectral data may be transmitted to a data center providing an interrogation device.

The invention thus includes a product which comprises a prescribed form of the described above authentication system wherein said authentication system comprises at least one population of fluorescent diamond particles wherein the wavelength, duration and intensity of the fluorescence emission of said particles is dependent on the wavelength, duration and intensity of the excitation energy.

In one embodiment, the diamond population in the prescribed form of authentication system used in the product is homogeneous; in another embodiment, the prescribed form comprises at least two different homogeneous populations of fluorescent diamond particles; wherein each different population has a unique fluorescence wavelength or intensity or duration or combination thereof. The product may include in the authentication system heterogeneous population of diamond particles, or may contain only said heterogeneous population. The product may have the populations of particles distributed throughout the composition, or if the composition is a solid, and the solid has a surface, the particles may be, but need not be, at the surface of the product.

In the latter case, the authentication system may optionally be distributed in a preset pattern, such as a number or letter. For products that are solids, the product may comprise a hydrophilic base in combination with a hydrophobic hygroscopic component, that optionally has been subjected to pressure to expel water from the hydroscopic component. Any of these products may be a pharmaceutical product. Any of these products may have associated therewith a code designating excitation wavelength(s), and/or duration(s) and/or intensity(ies) to be employed in authenticating the product, and the code for the excitation wavelength(s) and/or duration(s) and/or intensity(ies) may optionally be secret.

The invention also includes a method to authenticate a product which method comprises irradiating said product with excitation wavelength(s), duration(s) and intensity(ies) that generate(s) fluorescence from said population(s) of diamond particles and determining any fluorescence. In some instances, a spectrum comprising both wavelength and intensity and optionally duration of emissions from each population is determined.

The spectrum may also be transmitted to a data center or a detector programmed to recognize authentic spectra, which detector may be remote from the end user.

The invention is also directed to certain authentication systems comprising particulate diamond populations per se.

1. A product which comprises a prescribed form of an authentication system which system comprises at least one population of fluorescent diamond particles wherein the wavelength, duration and intensity of the fluorescence emission of said particles is dependent on the wavelength, duration and intensity of the excitation energy,
wherein said prescribed form consists of one homogeneous population of diamond particles, or
wherein said prescribed form consists of one heterogeneous population of said particles; or
wherein said prescribed form comprises at least two different homogeneous populations of fluorescent diamond particles; or
wherein said prescribed form comprises at least one population that is homogeneous and at least one population that is heterogeneous; and
wherein each different population has a unique fluorescence wavelength or intensity or duration or combination thereof.

2. The product of claim 1 which is associated with a code designating the excitation wavelength(s) and/or duration(s) and/or intensity(ies) that cause said population(s) to fluoresce, and/or designates the wavelength(s) and/or duration(s) and/or intensity(ies) of the fluorescent emission of the prescribed form.

3. The product of claim 2 wherein said code is secret and disclosed only to designated recipient(s).

4. The product of claim 1 wherein the prescribed form of authentication system is distributed throughout the product.

5. The product of claim 1 which is a powder, semisolid, emulsion or liquid.

6. The product of claim 1 which is a solid.

7. The product of claim 6 wherein the solid has a surface and the prescribed form of the authentication system is at the surface of the composition.

8. The product of claim 7 wherein the authentication system is distributed in a preset pattern.

9. The product of claim 6 which comprises a hydrophilic base in combination with a hydrophobic hygroscopic component.

10. The product of claim 9 which has been subjected to pressure to expel water from the hydroscopic component.

11. The product of claim 1 which is a pharmaceutical composition.

12. The product of claim 11 wherein the composition is a topical, an oral composition or a parenteral composition.

13. The product of claim 11 which is a solid oral dosage form.

14. The product of claim 1 which is a cosmetic, fragrance, ink, luxury item, food, textile, mechanical part, paint or a document of value.

15. A method to evaluate a test product for authenticity, which method comprises irradiating said product with (an) excitation wavelength(s), of certain duration(s) and intensity(ies) that generate(s) fluorescence from the population or populations of diamond particles in the prescribed form of the authentication system contained in the authentic product described in claim 1 and determining any fluorescence emitted from said test product; and
comparing said any fluorescence emitted from the test product with that characteristic of the prescribed form of authentication system that is contained in the authentic product.

16. The method of claim 15 wherein said determining and comparing is by eye.

17. The method of claim 15 wherein a spectrum comprising wavelength and intensity and optionally duration of the fluorescent emission of said test product is determined using a spectrophotometer or spectral data are determined with a detector.

18. The method of claim 17 wherein said spectral data are transmitted to an interrogation device for said comparing or spectral data determination and interrogation are included in the same apparatus.

19. The method of claim 18 wherein the interrogation device is programmed to compare spectral data of the test product to spectral data characteristic of the prescribed form of authentication system in the authentic product; wherein said comparing determines the product as authentic if the spectral data match and counterfeit if the spectral data do not match.

20. The method of claim 19 wherein said interrogation device is remote from the detector determining the spectral data.

21. The method of claim 19 wherein the interrogation and spectral data determination are included in the same apparatus.

22. A method for providing authentication to a product which method comprises combining said product with a prescribed form of an authentication system which system comprises at least one population of fluorescent diamond particles wherein the wavelength, duration and intensity of the fluorescence emission of said particles is dependent on the wavelength, duration and intensity of the excitation energy,
wherein said prescribed form consists of one homogeneous population of diamond particles or of one heterogeneous population of diamond particles; or
wherein said prescribed form comprises at least two different homogeneous populations of fluorescent diamond particles; or
wherein said prescribed form comprises at least one population that is homogeneous and at least one population that is heterogeneous; and
wherein each different population has a unique fluorescence wavelength or intensity or duration or combination thereof.

23. A prescribed form of an authentication system which system comprises at least one population of fluorescent diamond particles wherein the wavelength, duration and intensity of the fluorescence emission of said particles is dependent on the wavelength, duration and intensity of the excitation energy,
wherein said prescribed form comprises at least two different homogeneous populations of fluorescent diamond particles; or
wherein said prescribed form comprises at least one population that is homogeneous and at least one population that is heterogeneous; and
wherein each different population has a unique fluorescence wavelength or intensity or duration or combination thereof.
